SEQUENCE LISTING (2 sequences)

SEQ ID NO: 1 - DNA, 20 bp, Artificial
  primer sequence
agggttgataggttaagagc

SEQ ID NO: 2 - DNA, 20 bp, Artificial
  primer sequence
ccaacagctagttgacatcg

(19) United States
(12) Patent Application Publication
(10) Pub. No.: US 2009/0239256 A1
(43) Pub. Date: Sep. 24, 2009

(54) Detection of Micro-Organisms

(75) Inventors: Steven Giglio, West Croydon (AU); Paul Terence Monis, Hillbank (AU); Christopher Paul Saint, Largs Bay (AU)

Correspondence Address: LERNER, DAVID, LITTENBERG,;KRUMHOLZ & MENTLIK, 600 SOUTH AVENUE WEST, WESTFIELD, NJ, 07090, US

(73) Assignee: SOUTH AUSTRALIAN WATER CORPORATION, Adelaide (AU)

(21) Appl. No.: 11/921,812

(22) PCT Filed: Jun. 9, 2006
(86) PCT No.: PCT/AU2006/000802

§ 371 Date: Mar. 11, 2009

Related U.S. Application Data
(60) Provisional application No. 60/688,702, filed on Jun. 9, 2005.

(30) Foreign Application Priority Data
Jun. 9, 2005 (AU) 2005902996

(51) Int. Cl. C12Q 1/04; C12N 1/00; C12N 1/20

(52) U.S. Cl. 435/34; 435/243; 435/252.1; 435/253.1; 435/253.6

(57) ABSTRACT

The present invention relates to a method of detecting the presence of a viable micro-organism of interest in a sample containing a contaminating micro-organism.

This application claims priority from Australian Provisional Patent Application No. 2005902996 filed on 9 Jun. 2005 and U.S. Provisional Patent Application No. 60/688,702 filed on 9 Jun. 2005. The contents of both of these applications are to be taken as incorporated herein by this reference.

### FIELD OF THE INVENTION

The present invention relates to a method of detecting the presence of viable micro-organisms in a sample.

The present invention also relates to methods and liquid media for propagating micro-organisms.

### BACKGROUND OF THE INVENTION

There are many circumstances where there is a need to detect the presence of viable micro-organisms of interest in a sample containing other types of micro-organisms.

For example, there is often a need to detect the presence of viable bacteria in environmental water samples. However, one difficulty with the detection of some bacteria in such samples is the presence of other micro-organisms in the sample which interfere with the growth and detection of the bacteria of interest.

Legionella bacteria are an example of such a micro-organism. Legionella species are ubiquitous in distribution systems and environmental water sources as part of biofilms and sediments, and are often found to co-exist with other bacteria, protozoa and ciliates. The growth of Legionella bacteria is inhibited by the presence of other micro-organisms, and the presence of other micro-organisms can interfere with the detection of the bacteria.

Legionella species are responsible for sporadic and outbreak cases of atypical pneumonia (legionellosis) and a lesser form of infection known as Pontiac Fever, which is undiagnosed in many instances. Human activity has created a perfect environment for the growth and transmission of Legionella through such devices as cooling towers, spa pools, warm water systems, humidifiers and potting mixes. Aerosols are easily transported by wind and can cover a large area, potentially infecting numerous people. Since person-to-person transmission of legionellosis has never been documented, the measures for disease prevention have concentrated on eliminating the pathogen from water supplies.

Growth of Legionella in any medium is extremely difficult. Strict pH must be controlled as well as fulfilling the fastidious requirements of the organism. In addition, Legionella bacteria endogenously produce compounds toxic to the organism, and autoclaving of cultivation media also produces free radicals that are toxic to the bacteria. Growth of the bacteria is also susceptible to the production of toxic compounds produced by other micro-organisms.

Currently, detection of Legionella in water samples requires the use of traditional culture techniques which are both time consuming and expensive. Current culture techniques adopted by most reporting laboratories have turn around times in the range from 7 to 14 days, depending on the type of Legionella species present in the sample. The delay between sampling and confirmation is inadequate, as the incubation time for legionellosis is usually 2 to 10 days, the result in some cases being too late to prevent a potentially hazardous situation.

Adoption of alternative technologies, such as Polymerase Chain Reaction (PCR), into reporting laboratories has not occurred due to inherent limitations with the detection systems, particularly the inability to discriminate between viable and non-viable cells, and the inability to detect many micro-organisms in samples containing other types of micro-organisms.

Accordingly, there is a need for new methodologies that allow the detection of viable micro-organisms of interest in samples containing other types of micro-organisms, as occur for example in environmental samples. In addition, there is a need for new liquid media that can be used for the growth of fastidious micro-organisms such as Legionella.

The present invention relates to a method of detecting viable micro-organisms of interest in samples containing contaminating micro-organisms, and methods and liquid media for propagating micro-organisms.

A reference herein to a patent document or other matter which is given as prior art is not to be taken as an admission that that document or matter was known or that the information it contains was part of the common general knowledge as at the priority date of any of the claims.

### SUMMARY OF THE INVENTION

The present invention provides a method of detecting the presence of a viable micro-organism of interest in a sample, the method including the steps of:
(a) providing a sample including a micro-organism of interest and a contaminating micro-organism, wherein the micro-organism of interest has a lag phase greater than that of the contaminating micro-organism;
(b) inoculating the sample into a first liquid medium including an anti-microbial agent that kills actively dividing micro-organisms;
(c) incubating the first liquid medium so inoculated for a period of time sufficient to allow the contaminating micro-organism in the sample to reach log phase but not sufficient for the micro-organism of interest to reach log phase;
(d) inoculating a second liquid medium with an amount of the first liquid medium so incubated, the second medium including an agent that inhibits the activity of the anti-microbial agent;
(e) incubating the second liquid medium so inoculated for a period of time sufficient to allow growth of the micro-organism of interest; and
(f) detecting the presence of viable micro-organisms of interest in the sample by an increase in the value of a first parameter that correlates with the number of micro-organisms of interest detected in the second liquid medium incubated for a period of time to allow growth of the micro-organism of interest, as compared to the value of a second parameter that correlates with the number of micro-organisms of interest detected in any one or more of (i) the sample; (ii) the first liquid medium inoculated with the sample; and (iii) the first liquid medium incubated for a period of time sufficient to allow the contaminating micro-organism in the sample to reach log phase but not sufficient for the micro-organism of interest to reach log phase.

The present invention also provides a method of propagating a micro-organism of interest in a sample, the method including the steps of:
(a) providing a sample including a micro-organism of interest and a contaminating micro-organism, wherein the micro-organism of interest has a lag phase greater than that of the contaminating micro-organism;
(b) inoculating the sample into a first liquid medium including an anti-microbial agent that kills actively dividing micro-organisms;
(c) incubating the first liquid medium so inoculated for a period of time of sufficient to allow the contaminating micro-organism in the sample to reach log phase but not sufficient for the micro-organism of interest to reach log phase;
(d) inoculating a second liquid medium with an amount of the first liquid medium so incubated, the second medium including an agent that inhibits the activity of the anti-microbial agent; and
(e) incubating the second liquid medium so inoculated for a period of time sufficient to allow growth of the micro-organism of interest.

The present invention also provides a method of detecting the presence of a viable micro-organism of interest in a sample containing contaminating micro-organisms, the method including the steps of:
(a) using an anti-microbial agent to substantially reduce the number of viable contaminating micro-organisms in the sample without substantially reducing the number of viable micro-organisms of interest in the sample;
(b) inhibiting the activity of the anti-microbial agent after the reduction in the number of viable contaminating bacteria;
(c) propagating the viable micro-organisms of interest after the inhibition of the activity of the anti-microbial agent; and
(d) detecting the presence of viable micro-organisms of interest in the sample by an increase in the value of a first parameter correlated with the number of micro-organisms of interest detected in the sample after propagation, as compared to the value of a second parameter that correlates with the number of viable micro-organisms detected in the sample before propagation of the viable micro-organisms of interest.

The present invention also provides a method of propagating a micro-organism of interest in a sample containing contaminating micro-organisms, the method including the steps of:
(a) using an anti-microbial agent to substantially reduce the number of viable contaminating micro-organisms in the sample without substantially reducing the number of viable micro-organisms of interest in the sample;
(b) inhibiting the activity of the anti-microbial agent after the reduction in the number of viable contaminating bacteria; and
(c) propagating the viable micro-organisms of interest after the inhibition of the activity of the anti-microbial agent.

The present invention also provides a liquid micro-organism growth medium including an ion-exchange resin with either or both of the properties of an average pore radius of less than 200 Angstroms and an average surface area per gram of greater than 600 m²/g.

The present invention also provides a liquid medium including cysteine and an ion-exchange resin with either or both of the properties of an average pore radius of less than 200 Angstroms and an average surface area per gram of greater than 600 m²/g.

The present invention also provides a kit for growth and/or detection of a micro-organism, the kit including:
a liquid medium for growth of the micro-organism; and
an ion-exchange resin with either or both of the properties of average pore radius of less than 200 Angstroms and an average surface area per gram of greater than 600 m²/g, the ion exchange resin being separate to the liquid medium or combined with the liquid medium; the kit optionally further including instructions and/or reagents for the growth and/or detection of the micro-organism.

The present invention arises from studies into the detection of Legionella species in water samples. Existing technologies, such as polymerase chain reaction-based technologies, do not allow the detection of viable Legionella species in samples. In the present study, it has been found that liquid media containing certain ion-exchange resins are useful for promoting the growth of the bacteria. In addition, the use of such media in conjunction with a methodology that allows selective reduction of the numbers of contaminating bacteria, allows for the detection of viable Legionella bacteria in samples. The use of certain ion-exchange resins also does not interfere with assaying of the samples by some methods, such as real-time PCR.

Various terms that will be used throughout the specification have meanings that will be well understood by a skilled addressee. However, for ease of reference, some of these terms will now be defined.

The term “lag phase” as used throughout the specification is to be understood to mean that period of minimal cell division of a micro-organism following inoculation of the micro-organism into a liquid medium.

The term “log phase” as used throughout the specification is to be understood to mean that period of maximal constant growth rate of the micro-organism in a liquid medium.

In this regard, it will be understood that micro-organism growth in a liquid medium usually has at least three stages: lag phase, log phase and stationary phase. The lag phase is that phase required to adapt to the new environment. There is either no cell division or minimal cell division during this period. Once the micro-organism has adapted to a new environment, the log phase begins. During this phase the micro-organism is growing at the maximal rate allowed by the environmental conditions. The stationary phase is when the micro-organisms begin competing for a limited supply of nutrients, and there is little or no net increase in their number in the liquid medium.

The term “anti-microbial agent” as used throughout the specification is to be understood to mean an agent that kills, or substantially inhibits growth of, a micro-organism. In one form, the anti-microbial agent is an agent that kills an actively dividing micro-organism. In the case of anti-microbial agent that acts on bacteria, the term “anti-bacterial agent” may be used.

The term “amplification” or variants thereof as used throughout the specification is to be understood to mean the production of additional copies of a nucleic acid sequence. For example, amplification may be achieved using polymerase chain reaction (PCR) technologies (essentially as described in Dieffenbach, C. W. and G. S. Dveksler (1995) PCR Primer, a Laboratory Manual, Cold Spring Harbor Press, Plainview, N.Y.) or by other methods of amplification, such as rolling circle amplification on circular templates, such as described in Fire, A. and Xu, S-Q. (1995) Proc. Natl. Acad. Sci. 92:4641-4645.

### BRIEF DESCRIPTION OF THE FIGURES

FIG. 1 shows growth curves of Legionella pneumophila in broth culture containing charcoal (circles), pyruvate (triangles) or Diaion HP20 (crosses), and the growth curve of Legionella anisa in broth culture containing Diaion HP20 (squares).

FIG. 2 shows growth curves of Legionella pneumophila in broth culture containing Diaion HP20 (diamonds), Legionella anisa in broth culture containing Diaion HP20 (squares), Legionella pneumophila in broth culture containing Sepabeads 825 (triangles), Legionella anisa in broth culture containing Sepabeads 825 (crosses), Legionella pneumophila in broth culture containing a mixture of Diaion HP20 and Sepabeads 825 (asterisks), and Legionella anisa in broth culture containing a mixture of Diaion HP20 and Sepabeads 825 (circles).

FIG. 3 shows growth curves of various Legionella spp. in broth culture containing Sepabeads 825 medium:

FIG. 4 shows quantitation of viable and non-viable Legionella pneumophila in Diaion HP20 broth.

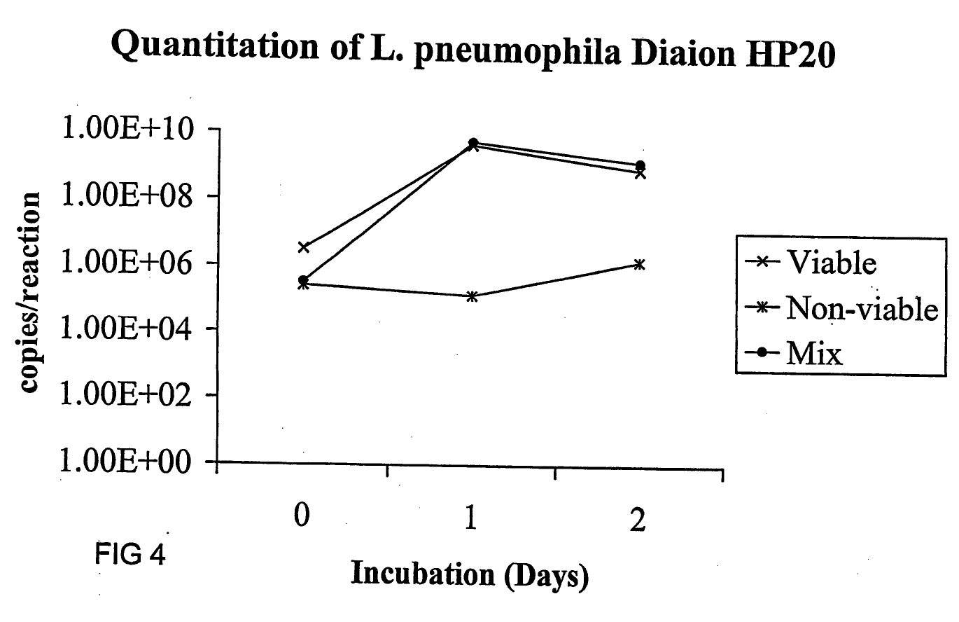

FIG. 5 shows a schematic of the methodology for Legionella detection in water samples.

### GENERAL DESCRIPTION OF THE INVENTION

As mentioned above, in one form the present invention provides a method of detecting the presence of a viable micro-organism of interest in a sample, the method including the steps of:
(a) providing a sample including a micro-organism of interest and a contaminating micro-organism, wherein the micro-organism of interest has a lag phase greater than that of the contaminating micro-organism;
(b) inoculating the sample into a first liquid medium including an anti-microbial agent that kills actively dividing micro-organisms;
(c) incubating the first liquid medium so inoculated for a period of time sufficient to allow the contaminating micro-organism in the sample to reach log phase but not sufficient for the micro-organism of interest to reach log phase;
(d) inoculating a second liquid medium with an amount of the first liquid medium so incubated, the second medium including an agent that inhibits the activity of the anti-microbial agent;
(e) incubating the second liquid medium so inoculated for a period of time sufficient to allow growth of the micro-organism of interest; and
(f) detecting the presence of viable micro-organisms of interest in the sample by an increase in the value of a first parameter that correlates with the number of micro-organisms of interest detected in the second liquid medium incubated for a period of time to allow growth of the micro-organism of interest, as compared to the value of a second parameter that correlates with the number of micro-organisms of interest detected in any one or more of (i) the sample; (ii) the first liquid medium inoculated with the sample; and (iii) the first liquid medium incubated for a period of time sufficient to allow the contaminating micro-organism in the sample to reach log phase but not sufficient for the micro-organism of interest to reach log phase.

This form of the present invention allows the detection of a viable micro-organism in a sample containing one or more types of other micro-organisms. This form of the present invention is useful, for example, for the detection of viable micro-organisms of interest in environmental samples, such as those from environmental water samples.

In this regard, it has been found that the detection of micro-organisms with a long lag phase in a sample containing other types of micro-organisms may be improved by taking advantage of the differential rate at which the micro-organisms reach log phase. Many of these other types of contaminating bacteria will have a lag phase shorter than that of the micro-organisms of interest.

By incubating the sample containing the micro-organisms of interest and the contaminating micro-organisms in a liquid medium for a period of time that allows the contaminating micro-organisms to begin growing, the number of these micro-organisms may be selectively reduced by an anti-microbial agent that only targets actively dividing cells. However, because the micro-organisms of interest have not yet begun actively dividing, they will not be killed by the anti-microbial agent.

Growth of the micro-organisms of interest may then be achieved by inhibiting the activity of the anti-microbial agent. Detection of a change in the population of the micro-organisms before and after growth is therefore indicative of the viability of the original inoculum.

The present invention may also be used for the propagation of micro-organisms.

Accordingly, in another form the present invention provides a method of propagating a micro-organism of interest in a sample, the method including the steps of:
(a) providing a sample including a micro-organism of interest and a contaminating micro-organism, wherein the micro-organism of interest has a lag phase greater than that of the contaminating micro-organism;
(b) inoculating the sample into a first liquid medium including an anti-microbial agent that kills actively dividing micro-organisms;
(c) incubating the first liquid medium so inoculated for a period of time sufficient to allow the contaminating micro-organism in the sample to reach log phase but not sufficient for the micro-organism of interest to reach log phase;
(d) inoculating a second liquid medium with an amount of the first liquid medium so incubated, the second medium including an agent that inhibits the activity of the anti-microbial agent; and
(e) incubating the second liquid medium so inoculated for a period of time sufficient to allow growth of the micro-organism of interest.

Examples of micro-organisms that have a long lag phase include bacterial species such as Legionella, mycoplasmas, ammonia oxidizing bacteria such as Nitrosonomas sp., Rhizobium sp., Treponema pallidum (the causative agent of syphilis) and other spirochaetes, Borrelia sp. (the causative agent of Lyme Disease), Bartonella sp. and Afipia sp. (the causative agent of cat scratch disease), Bordatella pertussis (the causative agent of whooping cough), Brucella sp., Francisella tularensis, Leptospira sp., Leptonema sp. and Mycobacterium sp.

In one form, the micro-organism of interest in the various forms of the present invention is a bacterium. For example, the micro-organism of interest may be a bacterium of the Legionella genus.

In this regard, examples of Legionella spp include L. pneumophila, L. anisa, L. micdadei, L. longbeachae, L. cincinatiensis, L. sainthelensis, L. saintcrusis, L. oakridgensis, L. birminghamensis, L. bozemanni, L. brunensis, L. cherrii, L. dumoffi, L. erythra, L. fairfieldensis, L. gestiae, L. gormanii, L. israelensis, L. jamestownensis, L. jordanis, L. lansingensis, L. londonensis, L. maceachernii, L. parisiensis, L. quateriensis, L. quinlivanni, L. rubriluscens, and L. tusconensis.

In one form, the contaminating micro-organism in the various forms of the present invention is a bacterium, such as a rapidly growing gram negative or gram positive bacterium.

Accordingly, in another form the present invention provides a method of detecting the presence of a viable bacterium of interest in a sample, the method including the steps of:
(a) providing a sample including a bacterium of interest and a contaminating bacterium, wherein the bacterium of interest has a lag phase greater than that of the contaminating bacterium;
(b) inoculating the sample into a first liquid medium including an anti-bacterial agent that kills actively dividing bacteria;
(c) incubating the first liquid medium so inoculated for a period of time sufficient to allow the contaminating bacteria in the sample to reach log phase but not sufficient for the bacteria of interest to reach log phase;
(d) inoculating a second liquid medium with an amount of the first liquid medium so incubated, the second medium including an agent that inhibits the activity of the anti-bacterial agent;
(e) incubating the second liquid medium so inoculated for a period of time sufficient to allow growth of the bacteria of interest; and
(f) detecting the presence of viable bacteria of interest in the sample by an increase in the value of a first parameter that correlates with the number of bacteria of interest detected in the second liquid medium incubated for a period of time to allow growth of the bacteria of interest, as compared to the value of a second parameter that correlates with the number of bacteria of interest detected in any one or more of (i) the sample; (ii) the first liquid medium inoculated with the sample; and (iii) the first liquid medium incubated for a period of time sufficient to allow the contaminating bacteria in the sample to reach log phase but not sufficient for the bacteria of interest to reach log phase.

In the case of Legionella bacteria, these bacteria typically have a long lag phase of approximately 8 to 24 hours in most liquid media, while most of the other contaminating bacteria in samples (such as those derived from environmental water samples) have a lag phase that is considerably shorter than that of the Legionella bacteria (generally in the range of 2 to 3 hours). Examples of bacteria with a lag phase that is considerably shorter than that of Legionella bacteria are as discussed previously.

Examples of samples containing the micro-organism of interest and contaminating micro-organisms of interest include water samples, liquid samples, soil samples, tissue or biological samples, samples containing animal or plant material generally, and other types of environmental samples.

In the case of the detection of viable Legionella bacteria, the sample in the various forms of the present invention is typically a water sample. In this case, the sample may be an environmental water sample, such as that obtained from a cooling tower, humidifier, air-conditioner, water storage facility, spa bath, shower, tap, soil, fountain, water cooled industrial saws or dental chair. It will also be appreciated that Legionella bacteria may also be present in other types of samples, including potting mixes or soil samples.

Inoculation of the sample into a first liquid medium may be accomplished by a suitable method known in the art. For example, in the case of a water sample, the sample (eg 1 ml) may be added directly to a suitable liquid culture medium (eg 50 ml).

However, it will also be appreciated that the sample for detection of micro-organisms of interest in the various forms of the present invention may be derived from a primary sample for analysis, and that the primary sample may be treated in some manner, so that the sample or material derived and/or extracted from the sample may be inoculated into the first medium.

For example, a soil sample containing Legionella bacteria may be first extracted with a suitable solution, broth etc to extract micro-organisms from the sample, and the extracted solution inoculated into the first liquid medium.

A suitable liquid medium for growth of Legionella spp. is a liquid medium including yeast extract, N-2-acetamido-2-aminoethane-sulfonic acid (ACES), α-ketoglutaric acid, potassium hydroxide, a detoxifying agent such as pyruvate or charcoal, ferric pyrophosphate and cysteine.

In regard to the use of a detoxifying agent as described above, and without being bound by theory, the use of a detoxifying agent for the growth of Legionella appears to facilitate growth of this bacterium by removing toxic compounds produced by the bacterium or from other sources, such as toxic compounds produced by other micro-organisms and/or by autoclaving. The use of detoxifying agent is also applicable to the growth of other fastidious bacteria.

An example of a media including pyruvate as a detoxifying agent is 1% yeast extract, 0.025% ferric pyrophosphate, 0.04% cysteine, 0.1% α-ketoglutaric acid, 1% N-2-acetamido-2-aminoethane-sulfonic acid, 0.28% potassium hydroxide and 0.1% sodium pyruvate.

An example of a media including charcoal as a detoxifying agent is 1% yeast extract, 0.025% ferric pyrophosphate, 0.04% cysteine, 0.1% α-ketoglutaric acid, 1% N-2-acetamido-2-aminoethane-sulfonic acid, 0.28% potassium hydroxide and 0.14% activated charcoal.

In one form, the detoxifying agent is an adsorbent material.

In this regard, it has been found that an ion-exchange resin is suitable as a detoxifying agent.

The ion-exchange resin may be composed of a matrix including aromatic, modified aromatic or methacrylic groups.

Aromatic type adsorbents are based on a cross-linked polystyrenic matrix, and are suitable for, for example, the extraction of antibiotic intermediates. Examples of such adsorbents include Diaion HP20 and HP21, Sepabeads SP825, SP850, SP70, and SP700.

Modified aromatic type adsorbents are based on a brominated aromatic matrix. This type of adsorbent is suitable for adsorption of organic substances of very low concentration or of highly hydrophilic substances.

Methacrylic type adsorbents are based on methacrylic ester copolymer. These types of adsorbent are suitable for adsorption of polyphenols and surfactants.

In one form, the ion-exchange resin is an aromatic type adsorbent.

A suitable concentration of a detoxifying agent may be selected, depending upon the characteristics of the micro-organism of interest. In the case of the use of Sepabeads 825 or 850, a suitable concentration is in the range from 5 to 15%.

A suitable characteristic of the ion-exchange resin for the growth of fastidious micro-organisms such as Legionella is an average surface area per gram of bead of greater than 600 m² and/or an average pore radius of less than 200 Angstroms (Å).

Accordingly, in another form the present invention provides a liquid micro-organism growth medium including an ion-exchange resin with either or both of the properties of an average pore radius of less than 200 Angstroms and an average surface area per gram of greater than 600 m²/g.

In the case of the average surface area of the ion exchange resin, typically the average surface area of the resin per gram is 1000 m²/g.

In the case of the average pore radius of the ion exchange resin, typically the average pore radius is 60 Angstroms or less, such as an average pore radius of 57 Angstroms or less. In some circumstances, an average pore radius of 38 Angstroms may be suitable.

Examples of ion-exchange resins with an average surface area per gram of greater than 600 m²/g and an average pore radius of less than 200 Angstroms are Sepabeads 825 (1000 m²) and Sepabeads 850 (1000 m²), both available from Mitsubishi Chemical Corporation. These ion-exchange resins are particularly suitable for analysis of samples by real-time PCR, as they do not float in the liquid medium and therefore may be readily removed from a sample for analysis.

An example of a suitable growth medium using an ion exchange resin is 1% yeast extract, 0.5% N-2-acetamido-2-aminoethane-sulfonic acid, 0.1% α-ketoglutaric acid, 0.2% potassium hydroxide, 0.04% cysteine and 0.025% ferric pyrophosphate and 5% ion-exchange resin (eg Diaion HP20, SP825, SP850).

Such mediums are particularly suitable for the growth of Legionella bacteria. Thus, the present invention also provides a liquid Legionella spp. growth medium including an ion-exchange resin with an average surface area per gram of greater than 600 m²/g and/or an average pore radius of less than 200 Angstroms.

The presence of cysteine in the liquid medium is suitable for the growth of Legionella bacteria.

Accordingly, in another form the present invention provides a liquid medium including cysteine and an ion-exchange resin with either or both of the properties of an average pore radius of less than 200 Angstroms and an average surface area per gram of greater than 600 m²/g.

A suitable concentration of cysteine in the liquid medium is from 0.1 g/l to 0.4 g/l.

The anti-microbial agent is an agent that kills, or substantially inhibits growth of, a micro-organism. Examples of anti-microbial agents include drugs, chemicals, small compounds and viruses.

Examples of anti-microbial agents that kill actively dividing bacteria (an “anti-bacterial agent”) in the various forms of the present invention include inhibitors of cell wall synthesis, which generally inhibit some step in the synthesis of bacterial peptidoglycan, such as penicillins, cephalosporins, carbapenems, monobactams, bacitracin, and glycopeptides.

Penicillins bind to and inhibit the carboxypeptidase and transpeptidase enzymes that are required for peptidoglycan biosynthesis. Examples of penicillins include amoxicillin, ampicillin, bacampicillin, carbenicillin, cloxacillin, dicloxacillin, flucloxacillin, methicillin, mezlocillin, nafcillin, oxacillin, penicillin G, penicillin V, piperacillin, pivampicillin, pivmecillinam, and ticarcillin.

Cephalosporins are also β-lactam antibiotics with a similar mode of action to penicillins. Examples include aztreonam, cefaclor, cefadroxil, cefamandole, cefazolin, cefdinir, cefepime, cefixime, cefoperazone, cefotaxime, cefotetan, cefoxitin, cefpodoxime, cefprozil, ceftazidime, ceftibuten, ceftizoxime, ceftriaxone, cefuroxime, cephalexin, cephapirin, and cephradine.

Two other classes of beta lactams are the carbapenems and monobactams. Examples of carbepenems include imipenem, meropenem and ertapenem. Examples of monobactams include aztreonam.

Bacitracin is a polypeptide antibiotic that prevents cell wall growth by inhibiting the release of the muropeptide subunits of peptidoglycan from the lipid carrier molecule that carries the subunit to the outside of the membrane.

Cycloserine inhibits the early stages of murein synthesis where D-alanyl-D-alanine is added to the growing peptide side chain.

Glycopeptides, such as vancomycin and teicoplanin, appear to inhibit both transglycosylation and transpeptidation reactions during peptidoglycan assembly. They bind to the muropeptide subunit as it is transferred out of the cell cytoplasm and inhibit subsequent polymerization reactions.

In the case of the removal of contaminating bacteria from a sample containing Legionella bacteria, the anti-microbial agent is typically vancomycin and/or cephamandole.

Alternatively, the anti-bacterial agent may be a bacteriophage that selectively infects dividing bacteria.

The anti-microbial agent in the various forms of the present invention will be used at a concentration that allows selective reduction of the number of the contaminating organisms as they proliferate, but does not substantially target the micro-organism of interest while it remains in lag phase. Appropriate concentrations of the anti-microbial agent are known in the art.

For example, a suitable concentration for the use of vancomycin in liquid media is 1 μg/ml. A suitable concentration of cephamandole is 1 μg/ml. These two antibiotics may both be included in the liquid medium.

The first liquid medium may also contain one or more agents that augment the action of the anti-microbial agent. For example, a cell membrane inhibitor such as polymyxin B may be used at a sub-lethal concentration for Legionella to aid in the removal of contaminating bacteria.

After inoculation of the sample into the first liquid medium, the medium is incubated under suitable conditions for a period of time sufficient to allow the contaminating micro-organism in the sample to reach log phase but not sufficient for the micro-organism of interest to reach log phase.

A suitable time may be selected depending upon the micro-organism of interest and the contaminating bacteria. In this regard, the period of time for many micro-organisms to reach log phase after the lag phase is known in the art. Alternatively, the period of time for a micro-organism to reach log phase may be readily determined by standard microbiological techniques known in the art.

For example, bacteria of the Legionella genus generally have a lag phase of greater than 12 hours, and in the range of approximately 12-36 hours. Other contaminating bacteria in environmental samples containing Legionella typically have a lag phase of 2-3 hours. Accordingly, incubation of the first liquid medium for 3 to 8 hours (for example) will allow the contaminating bacteria to reach log phase and therefore become susceptible to the action of the anti-microbial agent. However, Legionella bacteria present in the first liquid medium will not be killed by the anti-microbial agent, given that there has been insufficient time for the Legionella bacteria to reach log phase.

Accordingly, in another form the present invention provides a method of detecting the presence of viable Legionella bacteria in a sample, the method including the steps of:
(a) providing a sample including Legionella bacteria and contaminating bacteria;
(b) inoculating the sample into a first liquid medium including an anti-bacterial agent that kills actively dividing bacteria;
(c) incubating the first liquid medium so inoculated for 3 to 8 hours;
(d) inoculating a second liquid medium with an amount of the first liquid medium so incubated, the second medium including an agent that inhibits the activity of the anti-bacterial agent;
(e) incubating the second liquid medium so inoculated for a period of time sufficient to allow growth of the Legionella bacteria; and
(f) detecting the presence of viable Legionella bacteria in the sample by an increase in the value of a first parameter that correlates with the number of Legionella bacteria detected in the second liquid medium incubated for a period of time to allow growth of the Legionella bacteria, as compared to the value of a second parameter that correlates with the number of Legionella bacteria detected in any one or more of (i) the sample; (ii) the first liquid medium inoculated with the sample; and (iii) the first liquid medium incubated for a period of time sufficient to allow the contaminating bacteria in the sample to reach log phase but not sufficient for the Legionella bacteria to reach log phase.

In one form, the first liquid medium (step (c) as indicated above) is incubated for a period of 4 hours.

The second liquid medium is inoculated with an amount of the first liquid medium incubated for a period of time to selectively kill one or more contaminating micro-organisms present in the sample. The amount of the first liquid medium for inoculation into the second liquid medium is not particularly limited, and will generally depend upon the growth characteristics of the micro-organism of interest.

The second medium includes an agent that inhibits the activity of the anti-microbial agent.

The agent that inhibits the activity of the anti-microbial agent in the various forms of the present invention may function through a direct or indirect mechanism. For example, the agent may function directly by adsorbing the anti-microbial agent (eg activated charcoal or an ion-exchange resin), by binding to the anti-microbial agent, or by chemically reacting with the anti-microbial agent. Alternatively, the agent may inhibit the activity of the anti-microbial agent indirectly.

In one form, the agent acts by adsorbing the anti-microbial agent, such is the case for an ion-exchange resin. Details of suitable adsorbents, including suitable ion-exchange resins, are as discussed herein previously.

The ion-exchange resin may be, for example, Sepabeads 825 or 850. In this regard, it has been found that the use of a resin with beads that do not float in liquid medium is particular suitable for the analysis of samples by PCR, including real-time PCR, and the analysis of the samples by methods such as microscopy, flow cytometry, enzymatic assays, or by use of a biosensor.

A suitable concentration of the agent that inhibits the activity of the anti-microbial agent may be selected, depending upon the characteristics of the anti-microbial agent, the final concentration of active anti-microbial agent desired and the micro-organism of interest.

For example, for the growth of Legionella bacteria, a suitable concentration of Sepabeads 825 or 850 is 5%.

A suitable second liquid medium is 1% yeast extract, 0.5% N-2-acetamido-2-aminoethane-sulfonic acid, 0.1% α-ketoglutaric acid, 0.2% potassium hydroxide, 0.025% ferric pyrophosphate, 0.04% cysteine and 5% Sepabeads 825 or 5% Diaion HP20.

The second liquid medium is then incubated for a period of time sufficient to allow growth of the micro-organism of interest. This allows the assessment of viable micro-organisms in the sample.

An appropriate period of time may be selected, depending upon the growth characteristics of the particular micro-organism. For many micro-organisms these characteristics are known in the art. Alternatively, a suitable period of time may be selected by standard microbiological culture techniques.

The presence of micro-organisms of interest in the second liquid medium may be detected by a method known in the art.

It will be appreciated that the presence of viable micro-organisms of interest in the sample is detected by an increase in the value of a parameter that correlates with micro-organism number over the value of a parameter (the same or another parameter) after the micro-organism of interest has been propagated in the second liquid medium.

In this regard, the parameter that correlates with micro-organism number may be, for example, a direct measurement of the number of micro-organisms present.

For example, the micro-organism may be cultured on solid medium and the identification and number of the micro-organisms determined by a method known in the art.

In the case of Legionella, an aliquot may be drawn and serially diluted in phosphate buffered saline. Colony counts may then be determined by the spread plate method, in which 100 μl of serially diluted aliquot is spread onto BCYE agar plates using a sterile spreader. Plates are incubated at 35° C. for up to 7 days and colonies with classical Legionella morphology counted.

In this case, the first parameter and the second parameter are the same parameter. However, it will be appreciated that the first and second parameters in the various forms of the present invention need not be the same parameter.

A semi-quantitative method of detection may also be employed. For example, depending on the state, country, and specific legislation, action limits may apply to Legionella counts. As an example, AS/NZS 3666.3 “Performance based maintenance of cooling water systems” has a minimum detection limit of 10 cfu/mL, and an action limit of 1000 cfu/mL for Legionella counts, i.e. there is a 100-fold difference between action and no action limits. The use of this 100-fold difference can be used as a semi-quantitative tool for a body of water should this be required.

As an example, if the true Legionella count in a body of water was 1000 cfu/mL, then by performing the assay on the neat sample and a 100-fold dilution, it would be expected that both the neat sample and the 100-fold dilution would be positive; thus indicating the original count in the body of water was greater than or equal to 1000 cfu/mL. Similarly, if the true Legionella count in a body of water was 10 cfu/mL, it would be expected that the neat sample would be positive and the 100-fold dilution to be negative.

Alternatively, the parameter that correlates with micro-organism number may be an indirect measurement of the number of micro-organisms present.

For example, the micro-organism may be detected using an immunological detection system to detect the micro-organism of interest, such as enzyme-linked immunosorbent assay (ELISA), Western analysis, radioimmunoassay, immunofluorescence assay, or immunoenzyme assay.

Methods are known in the art for performing the above methods. For example, ELISA may be performed essentially as described in Clark B. and Engvall E. in “Enzyme Linked Immunosorbent Assay (ELISA): Theoretical and Practical Aspects in Enzyme-Immunoassay”, E. T. Maggio, ed., CRC Press, Inc., Boca Raton, Fla. (1980), pp. 167-179. In this regard, antibodies may be raised to a suitable protein encoded by the micro-organism by a method known in the art.

A suitable method for the detection of Legionella by an immunological method is as described in Berdal et al. (1979). “Detection of Legionella pneumonophila antigen in urine by enzyme-linked immunospecific assay.” J Clin Microbiol 9(5): 575-8.

Other methods of detecting micro-organisms include nucleic acid hybridization techniques, nucleic acid amplification techniques, latex agglutination assays, assays for enzymatic detection of a micro-organism, and flow cytometry.

Nucleic acid hybridization technologies are known in the art. Examples include Southern and Northern analysis (as described in Sambrook, J, Fritsch, E. F. and Maniatis, T. Molecular Cloning: A Laboratory Manual 2nd. ed. Cold Spring Harbor Laboratory Press, New York. 1989), and nucleic acid-chip technologies (as described in Fodor S P et al (1991) “Light-directed, spatially addressable parallel chemical synthesis” Science 251:767-773).

Examples of assays for enzymatic detection of micro-organisms include the use of enzymes specific for the micro-organism of interest. For example, the enzymatic activity of a specific enzyme from a micro-organism may be used, by coupling a substrate for the enzyme to a chromophore or fluorophore, and the cleavage of the substrate results in the production of colour or fluorescence which is indicative of the presence of the micro-organism.

Methods utilising flow cytometry may also be used. This method can also be used to discriminate between viable and non-viable cells by use of the appropriate detection reagents.

In the case of a nucleic acid amplification technology, one method of detecting the presence of micro-organism of interest is by the use of an amplification reaction, such as Polymerase Chain Reaction (PCR). In this case, the parameter that correlates with the number of micro-organisms is the amount of nucleic acid amplified. A suitable parameter is the number of copies of a particular gene detected by amplification.

Accordingly, in another form the present invention provides a method of detecting the presence of a viable Legionella bacterium in a sample, the method including the steps of:
(a) providing a sample including a Legionella bacterium and a contaminating bacterium;
(b) inoculating the sample into a first liquid medium including an anti-bacterial agent that kills actively dividing bacteria;
(c) incubating the first liquid medium so inoculated for a period of time sufficient to allow the contaminating bacteria in the sample to reach log phase but not sufficient for the Legionella bacteria to reach log phase;
(d) inoculating a second liquid medium with an amount of the first liquid medium so incubated, the second medium including an agent that inhibits the activity of the anti-bacterial agent;
(e) incubating the second liquid medium so inoculated for a period of time sufficient to allow growth of the Legionella bacteria; and
(f) detecting the presence of viable Legionella bacteria in the sample by an increase in the amount of a target Legionella gene detected by amplification in the second liquid medium incubated for a period of time to allow growth of the Legionella bacteria, as compared to the amount of a target Legionella gene detected by amplification in any one or more of (i) the sample; (ii) the first liquid medium inoculated with the sample; and (iii) the first liquid medium incubated for a period of time sufficient to allow the contaminating bacteria in the sample to reach log phase but not sufficient for the Legionella bacteria to reach log phase.

In the case of polymerase chain technologies, detection of the particular micro-organism may be, for example, by standard techniques or by real-time PCR technology. Polymerase chain reaction (PCR) technologies are generally as described in Dieffenbach, C. W. and G. S. Dveksler (1995) PCR Primer, a Laboratory Manual, Cold Spring Harbor Press, Plainview, N.Y.

DNA for amplification may be extracted from the micro-organism by a suitable method known in the art. Suitable primers and suitable reaction conditions may be used, depending upon the micro-organism of interest.

In the case of detection of bacteria, a suitable target sequence for amplification is the 16S rRNA gene. Detection of bacteria using the 16S rRNA gene is as described in Woese, C. R. and G. J. Olsen (1986). “Archaebacterial phylogeny: perspectives on the urkingdoms.” Syst Appl Microbiol 7: 161-77.

For detection of Legionella using amplification of the 16s rRNA gene, primers that amplify a portion of the 16S rRNA gene from approximately base 451 to base 837 of L. pneumophila ATCC 33152 may be used. The primers are as follows:

| | |
|---|---|
| Forward primer: | |
| 5′-AGGGTTGATAGGTTAAGAGC3′ | (SEQ ID No.1) |
| Reverse primer: | |
| 5′-CCAACAGCTAGTTGACATCG-3′. | (SEQ ID NO.2) |

As described above, the micro-organism may be detected by real-time PCR. In this case, preferably an ion-exchange resin in the second liquid medium is used that does not float, such as Sepabeads 825 or 850, so as to not interfere with the assay.

However, in the case that standard PCR is used, conditions for standard PCR to detect Legionella are as follows:

Five microliters of extracted template DNA may be used in a 50-μl reaction mixture that includes 1×PCR buffer (50 mM KCl, 10 mM Tris-HCl [pH 8.3]), 3 mM MgCl₂, 200 μM dATP, 200 μM dCTP, 200 μM dGTP, 400 μM dUTP, 1 μM reverse primer, 1 μM reverse primer, 2.8 U of AmpliTaq Gold per μl, and 1 U of uracil DNA glycosylase (UDG) (Life Technologies, Gaithersburg, Md.) per μl. Thermal cycling may be performed with a Perkin-Elmer GeneAmp PCR System 2400 (PE Applied Biosystems, Foster City, Calif.). Suitable cycling conditions are an initial incubation at 37° C. for 10 min to allow UDG degradation of uracil residues to prevent carryover amplicon contamination. After a 20-min hold at 95° C., 38 cycles consisting of 94° C. for 45 s, 57° C. for 45 s, and 72° C. for 45 may be followed by a final extension at 72° C. for 60 min, and the mixture held at this temperature until analysis.

As discussed above, the presence of viable micro-organisms in the sample is detected directly or indirectly by the increase in a parameter that correlates with the number of micro-organisms detected after growth of the micro-organisms in the second liquid medium over the value of a parameter that correlates with the number of micro-organisms detected before growth of the micro-organisms in the second liquid medium.

In this regard, the increase in the number of organisms detected may be an increase in the number of micro-organisms detected in the sample itself (an aliquot of the sample being held for analysis), and/or the number of micro-organisms detected in the first liquid medium after inoculation of the sample into the first liquid medium, and/or the number of micro-organisms detected in the first liquid medium after incubation to remove the contaminating bacteria.

For example, the increase in the number of micro-organisms may be measured by the number of micro-organisms detected in the second liquid medium after growth of the micro-organism of interest over the number of organisms detected in the first liquid medium after incubation to remove the contaminating bacteria.

An increase in micro-organism number is indicative of micro-organism growth and therefore the presence of viable micro-organisms in the sample.

The present invention also contemplates kits for the growth of a micro-organism, the kit including an ion-exchange resin for detoxifying a growth medium and/or removing anti-microbials.

In one form, the ion-exchange resin has an average pore radius of less than 200 Angstroms and/or an average surface area per gram of greater than 600 m²/g.

Accordingly, in another form the present invention provides a kit for growth and/or detection of a micro-organism, the kit including:
a liquid medium for growth of the micro-organism; and
an ion-exchange resin with either or both of the properties of an average pore radius of less than 200 Angstroms and an average surface area per grain of greater than 600 m²/g, the ion exchange resin being separate to the liquid medium or combined with the liquid medium; the kit optionally further including instructions and/or reagents for the growth and/or detection of the micro-organism.

In the case of Legionella bacteria, the instructions would, for example, provide directions to suitable growth conditions of one or more particular Legionella species, or instructions for the detection of the Legionella.

As discussed above, the kit may also further include additional reagents for the growth and/or detection of the micro-organism.

In the case of Legionella bacteria, the reagents and instructions would, for example, allow detection of the bacteria by polymerase chain reaction. Examples of reagents include the forward and reverse primers, as discussed previously. Examples of instructions include the cycling conditions for use with these primers, as previously discussed.

As discussed previously, the present invention allows the detection of viable micro-organisms in a sample by using an anti-microbial agent to substantially reduce the number of viable contaminating micro-organisms in the sample without substantially reducing the number of viable micro-organisms in the sample.

Accordingly, in another form the present invention provides a method of detecting the presence of a viable micro-organism of interest in a sample containing contaminating micro-organisms, the method including the steps of:
(a) using an anti-microbial agent to substantially reduce the number of viable contaminating micro-organisms in the sample without substantially reducing the number of viable micro-organisms of interest in the sample;
(b) inhibiting the activity of the anti-microbial agent after the reduction in the number of viable contaminating bacteria;
(c) propagating the viable micro-organisms of interest after the inhibition of the activity of the anti-microbial agent; and
(d) detecting the presence of viable micro-organisms of interest in the sample by an increase in the value of a first parameter correlated with the number of micro-organisms of interest detected in the sample after propagation, as compared to the value of a second parameter that correlates with the number of viable micro-organisms detected in the sample before propagation of the viable micro-organisms of interest.

It will be appreciated that as previously discussed herein, the sample will generally be derived from a primary sample containing the bacteria, such as an environmental water sample.

Examples of suitable micro-organisms of interest include bacterial species such as Legionella, mycoplasmas, ammonia oxidizing bacteria such as Nitrosonomas sp., Rhizobium sp., Treponema pallidum (the causative agent of syphilis) and other spirochaetes, Borrelia sp. (the causative agent of Lyme Disease), Bartonella sp. and Afipia sp. (the causative agent of cat scratch disease), Bordatella pertussis (the causative agent of whooping cough), Brucella sp., Francisella tularensis, Leptospira sp., Leptonema sp. and Mycobacterium sp.

In one form, the micro-organism of interest is a bacterium. For example, the micro-organism of interest may be a bacterium of the Legionella genus.

Examples of Legionella spp include L. pneumophila, L. anisa, L. micdadei, L. longbeachae, L. cincinatiensis, L. sainthelensis, L. saintcrusis, L. oakridgensis, L. birminghamensis, L. bozemanni, L. brunensis, L. cherrii, L. dumoffi, L. erythra, L. fairfieldensis, L. gestiae, L. gormanii, L. israelensis, L. jamestownensis, L. jordanis, L. lansingensis, L. londonensis, L. maceachernii, L. parisiensis, L. quateriensis, L. quinlivanni, L. rubriluscens, and L. tusconensis.

In one form, the contaminating micro-organism is a bacterium, such as a rapidly growing gram negative or gram positive bacterium.

Examples of anti-microbial agents are as previously discussed herein.

Methods of detection of the micro-organism are as previously discussed herein. In one form, the number of viable micro-organisms is determined by measuring the amount of a target gene amplified by PCR, as previously discussed.

The present invention is also useful for the propagation of micro-organisms.

Accordingly, in another form the present invention provides a method of propagating a micro-organism of interest in a sample containing contaminating micro-organisms, the method including the steps of:
(a) using an anti-microbial agent to substantially reduce the number of viable contaminating micro-organisms in the sample without substantially reducing the number of viable micro-organisms of interest in the sample;
(b) inhibiting the activity of the anti-microbial agent after the reduction in the number of viable contaminating bacteria; and
(c) propagating the viable micro-organisms of interest after the inhibition of the activity of the anti-microbial agent.

In one form, the present invention allows the detection of viable Legionella bacteria in a sample by substantially reducing the number of viable contaminating bacteria in the sample without substantially reducing the number of viable Legionella bacteria in the sample.

Accordingly, in another form the present invention provides a method of detecting the presence of viable Legionella bacteria in a sample containing contaminating bacteria, the method including the steps of:
(a) substantially reducing the number of viable contaminating bacteria in the sample without substantially reducing the number of viable Legionella bacteria in the sample;
(b) propagating the viable Legionella bacteria after the reduction in the number of viable contaminating bacteria; and
(c) detecting the presence of viable Legionella bacteria in the sample by determining the increase in the value of a first parameter correlated with the number of Legionella bacteria detected in the sample after propagation of the Legionella bacteria, as compared to the value of a second parameter correlated with the number of Legionella bacteria detected in the sample before propagation of the Legionella bacteria.

It will be appreciated that as previously discussed herein the sample will generally be derived from a primary sample containing the bacteria, such as an environmental water sample.

In one form, the number of viable Legionella bacteria is determined by measuring the amount of a target gene amplified by PCR, as previously discussed.

### DESCRIPTION OF THE PREFERRED EMBODIMENTS

Reference will now be made to experiments that embody the above general principles of the present invention. However, it is to be understood that the following description is not to limit the generality of the above description.

### Example 1

### Growth of Legionella in Various Media

A number of formulations of Legionella broths were tested to examine the requirement for detoxifying agents and cysteine for the growth of Legionella. L. pneumophila (a relatively fast growing Legionella) and L. anisa (a slower growing Legionella).

The details of broths A to G tested are as follows:

### Broth A

| | | |
|---|---|---|
| Proteose Peptone (Becton Dickinson) | 7 | g |
| Yeast Extract (Oxoid) | 5 | g |
| K₂HPO₄ (BDH) | 2 | g |
| KH₂PO₄ (BDH) | 0.5 | g |
| Ferric pyrophosphate (Sigma) | 0.125 | g |
| Cysteine (Sigma) | 0.2 | g |
| MQ Water | 500 | mL |

Ingredients were combined and pH adjusted to 6.9+/−0.1. The mixture was filter sterilised using Millipore 0.22 μm 500 mL filter apparatus, and 100 mL dispensed into sterile 120 mL blow mould containers.

### Broth B

| | | |
|---|---|---|
| Yeast Extract (Oxoid) | 5 | g |
| Ferric pyrophosphate (Sigma) | 0.125 | g |
| Cysteine (Sigma) | 0.2 | g |
| α-ketoglutaric acid (Sigma) | 0.5 | g |
| MQ Water | 500 | mL |

Ingredients were combined and pH adjusted to 6.9+/−0.1. The mixture was filter sterilised using Millipore 0.22 μm 500 mL filter apparatus, and 100 mL dispensed into sterile 120 mL blow mould containers.

### Broth C

| | | |
|---|---|---|
| Yeast Extract (Oxoid) | 5 | g |
| Ferric pyrophosphate (Sigma) | 0.125 | g |
| Cysteine (Sigma) | 0.2 | g |
| α-ketoglutaric acid (Sigma) | 0.5 | g |
| ACES (N-2-acetamido-2-aminoethane-sulfonic acid) | 5 | g |
| Potassium hydroxide (BDH) | 1.4 | g |
| Sodium pyruvate (Sigma) | 0.5 | g |
| MQ Water | 500 | mL |

ACES was from Sigma. Ingredients were combined and pH adjusted to 6.9+/−0.1. The mixture was filter sterilised using Millipore 0.27 μm 500 mL filter apparatus, and 100 mL dispensed into sterile 120 mL blow mould containers.

### Broth D

| | | |
|---|---|---|
| Yeast Extract (Oxoid) | 5 | g |
| Ferric pyrophosphate (Sigma) | 0.125 | g |
| Cysteine (Sigma) | 0.2 | g |
| α-ketoglutaric acid (Sigma) | 0.5 | g |
| ACES (Sigma) | 5 | g |
| Potassium hydroxide (BDH) | 1.4 | g |
| Charcoal (Sigma) | 0.7 | g |
| MQ Water | 500 | mL |

All ingredients except cysteine and ferric pyrophosphate were combined and pH adjusted to 7.1+/−0.1. The mixture was autoclaved for 15 min at 121° C. Cysteine and ferric pyrophosphate were added once the mixture had cooled to below 500. Approximately 100 mL was added to sterile 120 mL blow mould containers.

### Broth E

| | | |
|---|---|---|
| Yeast Extract (Oxoid) | 5 | g |
| Ferric pyrophosphate (Sigma) | 0.125 | g |
| Cysteine (Sigma) | 0.05 | g |
| α-ketoglutaric acid (Sigma) | 0.5 | g |
| ACES (Sigma) | 5 | g |
| Potassium hydroxide (BDH) | 1.4 | g |
| Charcoal (Sigma) | 0.7 | g |
| MQ Water | 500 | mL |

All ingredients except cysteine and ferric pyrophosphate were combined and pH adjusted to 7.1+/−0.1. The mixture was autoclaved for 15 min at 121° C. Cysteine and ferric pyrophosphate were added once the mixture had cooled to below 50° C. Approximately 100 mL was added to sterile 120 mL blow mould containers.

### Broth F

| | | |
|---|---|---|
| Yeast Extract (Oxoid) | 5 | g |
| Ferric pyrophosphate (Sigma) | 0.125 | g |
| Cysteine (Sigma) | 0.05 | g |
| α-ketoglutaric acid (Sigma) | 0.5 | g |
| ACES (Sigma) | 5 | g |
| Potassium hydroxide (BDH) | 1.4 | g |
| Charcoal (Sigma) | 0.7 | g |
| Bovine serum albumin (Sigma) | 0.5 | g |
| MQ Water | 500 | mL |

All ingredients except cysteine, ferric pyrophosphate and bovine serum albumin were combined and pH adjusted to 7.1+/−0.1. The mixture was autoclaved for 15 min at 121° C. Cysteine, ferric pyrophosphate and bovine serum albumin were added once the mixture had cooled to below 50° C. Approximately 100 mL was added to sterile 120 mL blow mould containers.

### Broth G

| | | |
|---|---|---|
| Yeast Extract (Oxoid) | 5 | g |
| Ferric pyrophosphate (Sigma) | 0.125 | g |
| Cysteine (Sigma) | 0.05 | g |
| α-ketoglutaric acid (Sigma) | 0.5 | g |
| ACES (Sigma) | 5 | g |
| Potassium hydroxide (BDH) | 1.4 | g |
| Charcoal (Sigma) | 0.7 | g |
| MQ Water | 500 | mL |

All ingredients except cysteine and ferric pyrophosphate were combined and pH adjusted to 7.1+/−0.1. The mixture was autoclaved for 15 min at 121° C. Cysteine and ferric pyrophosphate were added once the mixture had cooled to below 50° C. The solution was then spun at 3500×g for 30 minutes Approximately 100 mL of the supernatant was added to sterile 120 mL blow mould containers.

The above broths were seeded with test Legionella to a final concentration of approximately 10³-10⁶ cfu/mL and incubated at 35° C. with shaking at 180 rpm for 48 hours. Aliquots were drawn at nominated time intervals and serially diluted in phosphate buffered saline. Colony counts were determined by the spread plate method. Briefly, 100 μl of serially diluted aliquot was spread onto BCYE agar plates using a sterile spreader. Plates were incubated at 35° C. for up to 7 days and colonies with classical Legionella morphology were counted and enumerated.

Mean generation times were calculated and are presented in Table 1.

TABLE 1
Mean generation times Legionella in various broths

Mean generation Times (min)

| Legionella broth | L. pneumophila sero group 1 | L. anisa |
|---|---|---|
| A | 178 | — |
| B | 166 | — |
| C | 84 | 245 |
| D | 86 | 121 |
| E | 72 | — |
| F | 85 | 175 |
| G | 78 | — |

— not available

A notable difference between broth A, B and the remainder broths was found for growth of L. pneumophila. A notable difference was also found between broth C and others for L. anisa growth, due to the presence of the detoxifying agents tested (A had a mixture, F had albumin and C had sodium pyruvate). Broths E and G consisted of a 4-fold difference in the amount of cysteine available and show that the effect of cysteine on growth rate in negligible.

These data indicate that charcoal as a detoxifying agent is superior to others initially trialled. However, detection of Legionella using real-time PCR from these broths was not able to be performed due to inhibition by charcoal.

### Example 2

### Use of Adsorbent Resins

Use of an adsorbent resin was trialled as an alternative detoxifying agent to charcoal. A Diaion HP20® resin broth formulation was initially trialled and compared to a charcoal based broth and a pyruvate based broth. The broths compared were as follows:

### Charcoal Broth

| | | |
|---|---|---|
| Yeast Extract (Oxoid) | 5 | g |
| Ferric pyrophosphate (Sigma) | 0.125 | g |
| Cysteine (Sigma) | 0.2 | g |
| α-ketoglutaric acid (Sigma) | 0.5 | g |
| ACES (Sigma) | 5 | g |
| Potassium hydroxide (BDH) | 1.4 | g |
| Charcoal (Sigma) | 0.7 | g |
| MQ Water | 500 | mL |

All ingredients except cysteine and ferric pyrophosphate were combined and pH adjusted to 7.1+/−0.1. The mixture was autoclaved for 15 min at 121° C. Cysteine and ferric pyrophosphate were added once the mixture had cooled to below 50°. Approximately 100 mL was added to sterile 120 mL blow mould containers.

### Pyruvate Broth

| | | |
|---|---|---|
| Yeast Extract (Oxoid) | 5 | g |
| Ferric pyrophosphate (Sigma) | 0.125 | g |
| Cysteine (Sigma) | 0.2 | g |
| α-ketoglutaric acid (Sigma) | 0.5 | g |
| ACES (Sigma) | 5 | g |
| Potassium hydroxide (BDH) | 1.4 | g |
| Sodium pyruvate (Sigma) | 0.5 | g |
| MQ Water | 500 | mL |

Ingredients were combined and pH adjusted to 6.9+/−0.1. The mixture was filter sterilised using Millipore 0.22 μm 500 mL filter apparatus, and 100 mL dispensed into sterile 120 mL blow mould containers.

### Diaion HP20 Broth

| | | |
|---|---|---|
| Yeast Extract Agar (Oxoid) | 10 | g |
| ACES buffer (Sigma) | 5 | g |
| α-ketoglutaric acid (Sigma) | 1 | g |
| Potassium hydroxide (BDH) | 2 | g |
| Diaion HP20 (Mitsubishi Chemicals) | 50 | g |
| MQ Water | make up to 1 | L |
| Cysteine (Sigma) | 0.4 | g |
| Ferric pyrophosphate (Sigma) | 0.25 | g |

All ingredients except cysteine and ferric pyrophosphate were combined and pH adjusted to 7.1+/−0.1. The mixture was autoclaved for 15 min at 121° C. Cysteine and ferric pyrophosphate were added once the mixture bad cooled to below 50° C. Approximately 100 mL was added to sterile 120 mL blow mould containers.

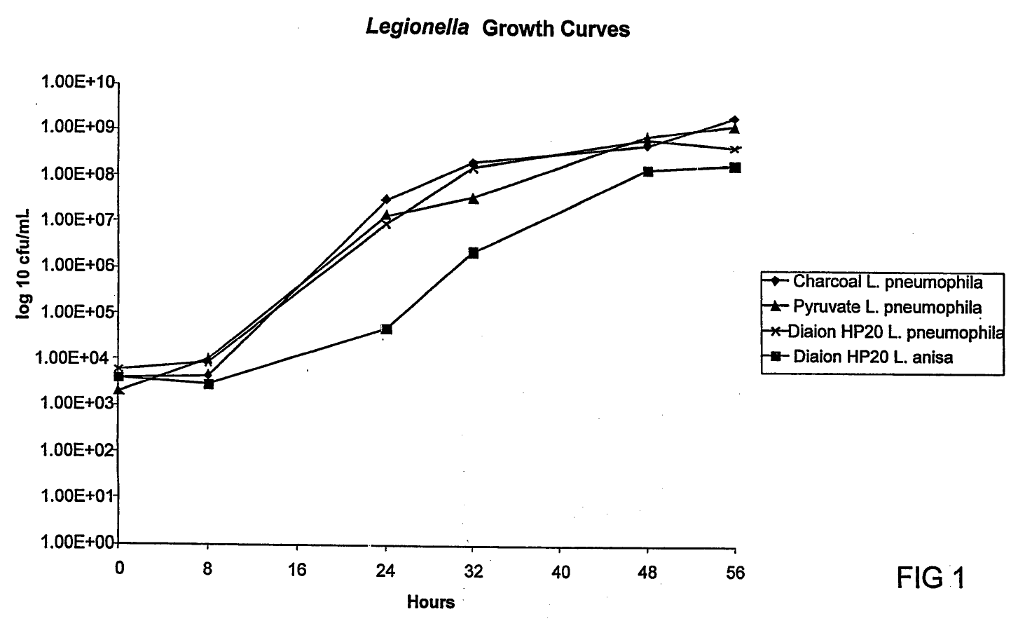

FIG. 1 shows that the resin performs as well as the charcoal based broth. Similarly, detection of L. anisa closely follows the growth curve of L. pneumophila. The mean generation time for L. anisa in this experiment was 95 minutes, which is slightly less than the times seen in Table 1. L. anisa typically takes 5 days to form visible colonies on agar BCYE agar, compared to L. pneumophila which takes 3-4 days, and it appears that in liquid culture the difference in time for detection of traditionally slower growing Legionella and L. pneumophila can be significantly reduced.

### Example 3

### Use of Sepabeads 825® Resin

Diaion HP20 resin is buoyant in the media and interfered with taking aliquots from the reaction vessel and was considered to be likely to interfere with PCR should a bead be placed in the reaction vessel.

Sepabeads 825® resin is larger than Diaion HP200, the beads do not float and the beads have the additional property of a larger surface area per gram (SP825—average pore radius 57 Angstroms, average surface area per gram of bead 1000 m²; Diaion HP20— average pore radius 200 Angstroms, average surface area per gram of bead 600 m²). Combined, these properties were considered to make Sepabeads® an alternative to trial not only as a detoxifying compound, but also as a growth accelerant.

Three Legionella broth formulations containing 5% Sepabeads 825®, Diaion HP20®, and a mixture of both were inoculated with L. pneumophila and L. anisa and growth curves were generated as previously described.

The details of the broths were as follows:

### SP825 Broth

| | | |
|---|---|---|
| Yeast Extract Agar (Oxoid) | 10 | g |
| ACES buffer (Sigma) | 5 | g |
| α-ketoglutaric acid (Sigma) | 1 | g |
| Potassium hydroxide (BDH) | 2 | g |
| SP825 (Mitsubishi Chemicals) | 50 | g* |
| MQ Water | make up to 1 | L |
| Cysteine (Sigma) | 0.4 | g |
| Ferric pyrophosphate (Sigma) | 0.25 | g |

All ingredients except cysteine and ferric pyrophosphate were combined and pH adjusted to 7.1+/−0.1. The mixture was autoclaved for 15 min at 121° C. Cysteine and ferric pyrophosphate were added once the mixture had cooled to below 50° C. Approximately 100 mL (or 50 mL for environmental field trials) was added to sterile 120 mL blow mould containers

### Diaion HP20 Broth

| | | |
|---|---|---|
| Yeast Extract Agar (Oxoid) | 10 | g |
| ACES buffer (Sigma) | 5 | g |
| α-ketoglutaric acid (Sigma) | 1 | g |
| Potassium hydroxide (BDH) | 2 | g |
| Diaion HP20 (Mitsubishi Chemicals) | 50 | g |
| MQ Water | make up to 1 | L |
| Cysteine (Sigma) | 0.4 | g |
| Ferric pyrophosphate (Sigma) | 0.25 | g |

All ingredients except cysteine and ferric pyrophosphate were combined and pH adjusted to 7.1+/−0.1. The mixture was autoclaved for 15 min at 121° C. Cysteine and ferric pyrophosphate were added once the mixture had cooled to below 50° C. Approximately 100 mL was added to sterile 120 mL blow mould containers.

### Mixture

| | | |
|---|---|---|
| Yeast Extract Agar (Oxoid) | 10 | g |
| ACES buffer (Sigma) | 5 | g |
| α-ketoglutaric acid (Sigma) | 1 | g |
| Potassium hydroxide (BDH) | 2 | g |
| SP825 (Mitsubishi Chemicals) | 25 | g |
| Diaion HP20 (Mitsubishi Chemicals) | 25 | g |
| MQ Water | make up to 1 | L |
| Cysteine (Sigma) | 0.4 | g |
| Ferric pyrophosphate (Sigma) | 0.25 | g |

All ingredients except cysteine and ferric pyrophosphate were combined and pH adjusted to 7.1+/−0.1. The mixture was autoclaved for 15 min at 121° C. Cysteine and ferric pyrophosphate were added once the mixture had cooled to below 50° C. Approximately 100 mL was added to sterile 120 mL blow mould containers.

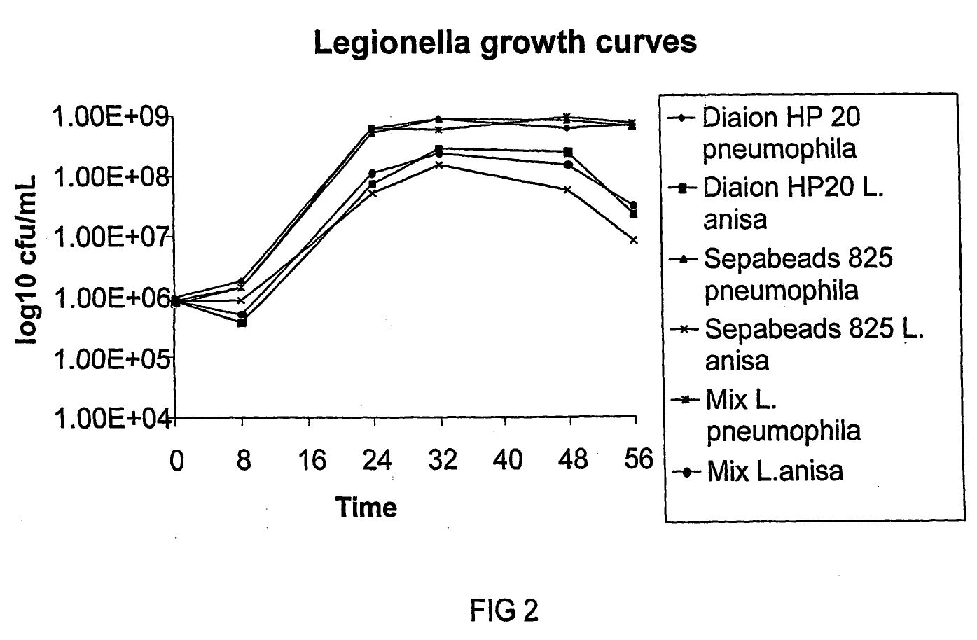

The data is shown in FIG. 2, and the mean generation times provided in Table 2.

TABLE 2
Mean generation times of Legionella broths incorporating 5%
Diaion HP20 ® and Sepabeads 825 ®

Mean generation time (min)

| Legionella Broth | L. pneumophila serogroup 1 | L. anisa |
|---|---|---|
| Diaion | 120 | 125 |
| Sepabeads | 111 | 139 |
| Mix of Diaion + | 106 | 125 |
| Sepabeads | | |

As can be seen, the differences between mean generation times of control organisms was negligible (Table 2), and the growth curve characteristics observed were similar (FIG. 2). The results indicate that the use of Sepabeads 825® over Diaion HP20® may be a suitable alternative for use in broth formulations.

### Example 4

### Legionella Species Growth Characterisation in Broths Incorporating Diaion and Sepabeads 825®

Having demonstrated the ability to support the growth of control Legionella strains with resin present, a suite of Legionella species were monitored for the ability to grow in the formulations. Spectrophotometric absorbance at 600 nm (OD₆₀₀) was used instead of serial dilution and counting of organisms. Legionella was spiked into the broths to obtain a final concentration of 5-log/mL. Growth was monitored over time by aliquoting broths and measuring absorbance at 600 nm.

The disadvantage of this approach was that it lacked sensitivity because a 1-log increase after lag phase could not be detected (i.e. an increase from 5-log to 6-log), and therefore the exact moment of entering log phase could not be determined.

The results of Legionella growth characteristics are presented in Table 3.

TABLE 3
Time to reach log phase for Legionella in broths

| | | | Sepabeads |
|---|---|---|---|
| | | Diaion HP20 | 825 |
| | | time to log | time to log |
| | | phase | phase |
| Legionella culture | ATCC | (hours) | (hours) |
| L. pneumophila serogroup 1 | 43111 | 12-24 | 12-24 |
| L. pneumophila serogroup | 43736 | 24-36 | 20-24 |
| 13 | | | |
| L. pneumophila serogroup 9 | 35298 | na | 12-24 |
| L. pneumophila serogroup 6 | 33215 | 22-36 | 12-24 |
| L. anisa | 35292 | 12-24 | 12-24 |
| L. micdadei | 33218 | 22-36 | 12-24 |
| L. longbeachae serogroup 1 | 33462 | 22-36 | 22-36 |
| L. longbeachae serogroup 2 | 33484 | 22-36 | 12-24 |
| L. cincinatiensis | 43753 | 48-52 | 12-24 |
| L. sainthelensis | 35248 | 48-52 | 12-24 |
| L. saintcrusis | 43119 | na | 12-24 |
| L. oakridgensis | 33761 | 12-24 | 12-24 |
| L. birminghamensis | 43702 | 12-24 | 28-32 |
| L. bozemanni serogroup 1 | 33217 | 12-24 | 12-24 |
| L. bozemanni serogroup 2 | 35545 | 22-36 | 12-24 |
| L. brunensis | 43878 | 22-36 | 12-24 |
| L. cherrii | 35252 | na | 12-24 |
| L. dumoffi | 33279 | 22-36 | 12-24 |
| L. erythra | 35303 | 12-24 | 12-24 |
| L. fairfieldensis | 49588 | 22-36 | 35-48 |
| L. gestiae | wild | na | 48-52 |
| L. gormanii | 33297 | 12-24 | 12, 24 |
| L. israelensis | 43119 | 12-24 | n/a |
| L. jamestownensis | 35298 | 12-24 | 12-24 |
| L. jordanis | 33623 | 12-24 | 12-24 |
| L. lansingensis | 49751 | 12-24 | 12-24 |
| L. londonensis | wild | 48-52 | 24-28 |
| L. maceachernii | 35300 | 12-24 | 12-24 |
| L. parisiensis | 35299 | 12-24 | 12-24 |
| L. quateriensis | wild | na | 12-24 |
| L. quinlivanni | 43830 | 12-24 | 12-24 |
| L. rubriluscens | 35304 | 24-36 | 12-24 |
| L. tusconensis | 49180 | 24-36 | 24-36 |

na—not available

Both adsorbent resins were able to support the growth of Legionella. The time to reach log phase was species and resin dependant. Notable differences in the time to reach log phase were seen with L. londonensis, L. cincinnatiensis, L. sainthelensis, and L. pneumophila serogroup 6, where Sepabead 825® performance was superior. Similarly the growth of L. birminghamensis was superior in Diaion HP20® formulations. The differences observed may be due to the ability of the particular resin to adsorb a specific toxic radical produced by the Legionella strain in question and/or the increase in surface area provided by the Sepabeads 825®.

Additional testing of Sepabeads 850® (average pore radius 38 Angstroms, average surface area per gram of bead 1000 m²), which has a smaller pore size than Sepabeads 825®, was performed on selected strains and compared to Sepabeads 825®. The results suggested that Sepabeads 850® is capable of supporting growth of Legionella strains tested (n=4) and that Sepabeads 825® seemed to accelerate the growth of L. bozemanii serogroup 2 and L. dumoffi when compared to the Sepabeads 850® resin.

These results are the first such characterisation of a wide range of Legionella in broth cultures. These and previous data indicate that Legionella species typically have a lag phase of 8-24 hours and quantitative changes in population can be detected (either by traditional colony counting or molecular) in less than one day. Detection of a change in population is important because the difference in population is indicative of growth and therefore cell viability of the original inoculum.

### Example 5

### Real Time PCR Detection

One of the limitations of using PCR for detection of organisms is the inability to distinguish between DNA from viable bacteria and DNA from non-viable bacteria. In the case of Legionella, this would cause a false positive interpretation of system maintenance procedures and unnecessary remedial actions would be administered. By combination of the Legionella broths and Real Time PCR, a method was developed to detect the presence of viable cells by measuring a change in cell numbers (copies of the target gene) over time.

To demonstrate the ability to detect viable cells amongst non-viable cells, a culture of L. pneumophila serogroup 1 was killed in 70% ethanol for 30 minutes and inoculated into a Legionella broth containing Diaion® HP20. Additionally, a viable culture was inoculated as well as a mixture of viable and non-viable ethanol killed bacteria. A ‘base count’ of DNA was taken at time zero, and then after 1 and 2 days of incubation at 35° C. Legionella DNA was quantitated by taking a ‘base count’ of DNA using a 16S rRNA gene assay described by Cloud et al (2000) J Clin Microbiol. 38(5):1709-12 on a Rotorgene 2000 (Corbett Research, Sydney, Australia). This assay has been previously validated in-house for a large number of environmental Legionella isolates (data not shown).

Briefly, Legionella pneumophila serogroup 1 viable and non-viable counts were determined by flow cytometric counts using a FACSCalibur flow cytometer and BacLight® Live-Dead kit as per manufacturer's instructions. After confirming the status of the inoculated bacteria by this method, broths were inoculated with Legionella (viable, non viable, and mix) to a final concentration of approximately 10⁶ cfu/mL and broths incubated at 35° C. for 48 hours and shaken at 180 rpm. Aliquots were drawn at nominated time intervals and assayed by real-time PCR, as follows:

Legionella 16S rRNA PCR was performed as described previously by Cloud et al, except that AmpliTaq Gold was used as the DNA polymerase, the reaction volume was 25 μl and the cycling conditions were changed to an initial hold at 95° C. for 10 min, followed by 40 cycles consisting of 94° C. for 20 s, 60° C. for 20 s, and 72° C. for 25 s., 5 μL of template DNA was used in a 25 μl reaction mixture that included 1×PCR buffer II (Applied Biosystems, New Jersey USA), 2.5 mM MgCl₂, 200 μM dNTP mix (Promega Corporation, Madison USA), 0.5 μM each of forward and reverse primer, 3.34 μM SYTO9 (Molecular Probes, OR), and 1 U AmpliTaq Gold (Applied Biosystems, New Jersey USA). All reactions were carried out in a RotorGene 3000 (Corbett Research, Sydney, Australia) with data acquisition at 72° C. on the FAM channel (excitation at 470 nm, detection at 510 nm n) at a gain of 5. Amplification take-off (defined as the cycle where exponential amplification is occurring) was determined using the comparative quantitation feature of the RotorGene software for the amplification data acquired at a gain of 5. Following amplification, melting curve data were acquired on the FAM channel (at gains of 2 and 5) using a ramping rate of 1° C./60 s from 75° C. to 95° C. The differentiated data were analysed using RotorGene software with the digital filter set as ‘none’. When required, samples were analysed by 1% agarose gel electrophoresis with the addition of Gelstar nucleic acid stain (Cambrex Bio Science, Rockland Inc) using standard methods, with the incorporation of DNA standards.

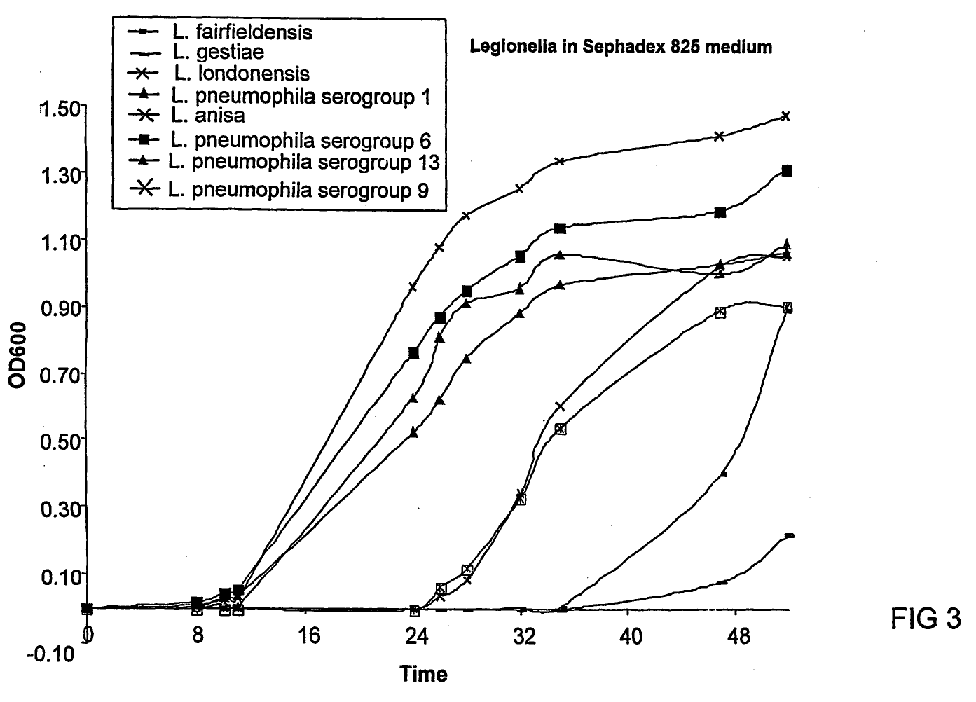

FIG. 3 shows an increase in Legionella numbers after 1 day incubation. The non-viable (ethanol killed) levels remained unchanged. A quantitative change of the mixture of viable and non-viable Legionella was detected, indicating that the increase in numbers detected must be due to the presence of the viable cells only. This approach can therefore be used to detect the presence of viable bacteria in a mixed population of viable and non-viable cells.

### Example 6

### Sepabead 825® Broth Performance in PCR

Having demonstrated in principle that discrimination between viable and non-viable population is possible, the suitability of using the Sepabeads 825® formulation in PCR was evaluated.

Serial dilution of L. pneumophila serogroup 1 was performed using the Sepabeads® broth as a diluent. Organism concentration ranged from 10⁶ cfu/mL to 10² cfu/mL. A 5 μl aliquot of each cell dilution was used as template DNA for the PCR reaction, as described previously. Legionella was detected across the dilution range (data not shown), indicating that the Sepabeads 825® formulation does not affect the detection of low levels of Legionella in solution.

### Example 7

### Formulation of Pre Enrichment/Selective Broth for Use in Detecting Legionella

Preliminary use of the Sepabeads 825® broth alone or in conjunction with pre-treating water samples (with acid or heat as described in AS/NZS 3896:1998) was unsuccessful in removing background microbial flora (data not shown). The problem was attributed to one or more of the overgrowth by rapidly growing bacteria originally present in the water sample that competed with Legionella for nutrients in the broth, obscured the detection of Legionella due to competitive effects during PCR, and the production of toxic by-products by these overgrowing organisms.

In order to address this issue, a new broth (PTB) was formulated to deal with these contaminating bacteria. The PTB formulation is based closely on the Sepabeads825® formulation except that it uses pyruvate as a detoxifying agent (instead of Sepabeads) and that it also includes the addition of anti-microbials.

### PTB

| | | |
|---|---|---|
| Yeast Extract Agar (Oxoid) | 10 | g |
| ACES buffer (Sigma) | 5 | g |
| α-ketoglutaric acid (Sigma) | 1 | g |
| Potassium hydroxide (BDH) | 2 | g |
| Pyruvic acid (Sigma) | 1 | g |
| Cysteine (Sigma) | 0.4 | g |
| Ferric pyrophosphate (Sigma) | 0.25 | g |
| Vancomycin (Sigma) | 1 | mg |
| Cephamandole (Sigma) | 1 | mg |
| Polymixin B (Sigma) | 50000 | U |
| MQ water | make up to 1 | L |

Ingredients for PTB were combined and pH adjusted to 6.9+/−0.1. The mixture was filter sterilised using Millipore 0.22 μm 500 mL filter apparatus, and 50 mL dispensed into sterile 120 mL blow mould containers.

Subsequent trials have demonstrated that autoclaving the above recipe (minus cysteine, ferric pyrophosphate, vancomycin, cephamandole, and polymyxin B) with the addition of the excluded components at a temperature less than 50° C. does not affect the performance of the media.

The data is shown in Table 4.

TABLE 4
PTB effect on organism number

| | Count at time | Count after |
|---|---|---|
| | zero | incubation |
| Organism | (cfu/mL) | (cfu/mL) |
| L. pneumophila serogroup 1 | 3100 | 5100 |
| L. anisa | 1400 | 5500 |
| S. aureus | 1370 | 220 |
| E. faecalis | 300 | 130 |
| E. coli | 6200 | <10 |

The time required for Legionella to reach lag phase is approximately 8-12 hours or greater, whilst the typical lag phase of the other organisms tested are 2-3 hours. At the completion of lag phase, organisms enter a phase of replication (log phase) and may be subject to the effects of certain anti-microbials action that target cell well synthesis e.g. (cephamandole and vancomycin). Any interference with cell wall synthesis will result in incomplete synthesis and hence cell destruction. Additionally, the use of cationic detergents (e.g. polymixin B) at certain concentrations can act on the phospholipid bi-layers of some organisms and result in cell death. In PTB, the end result is that these anti-microbials will not affect Legionella but will have an effect on a large number of other micro-organisms, resulting in a significant reduction of background flora which will allow the detection of Legionella in the Sepabeads 825® broth. As shown in Table 4, there is 83% removal of S. aureus, 57% removal of E. faecalis, and greater than 99% removal of E. coli. The numbers of Legionella have increased slightly and are not due to cell growth, but more likely due to the inherently large variation traditionally seen when determining Legionella counts by the standard plate method (AS/NZS 3896:1998).

The Sepabeads 825® broth allows removal of the anti-microbials in PTB. This is demonstrated by the following experiment:

Sepabeads 825 broths were prepared as previously described with the addition of 8, 16, and 32 mg cycloheximide (an antifungal); 0.1, 0.3, and 0.5 mg vancomycin; and 5000, 15000, and 25000 IU of polymyxin B. Anti-microbials present at these levels is in excess of what is required to kill the control organisms used. Standard Sepabeads 825 broth (i.e. no anti-microbials) were also prepared. E. coli was inoculated into the broths containing vancomycin and polymyxin, and the standard broth (control broth) at approximately 10³⁻⁴ cfu/mL. Similarly, C. albicans was inoculated into the cycloheximide broths and the standard broth (control broth). All broths were incubated at 35° C. for 48 hours and shaken at 180 rpm. Aliquots were drawn at nominated time intervals and OD₆₀₀ measurements taken. There growth profiles of the organisms in both the standard broth and broth with anti-microbials were not different, suggesting the adsorption of the anti-microbials by the Sepabeads 825.

### Example 8

### Final Methodology for Detection of Legionella in Water Samples

The final methodology for detection of viable Legionella in a water sample did not involve any of the traditional pre-treatments as outlined in AS/NZS 3896:1998, the current standard. Instead, 1 mL of water sample was inoculated into 50 mL PTB and incubated at 35° C. for 4 hours, shaken at 180 rpm. The entire contents of PTB were aseptically transferred to 15% Sepabeads 825® broth. An aliquot of the resultant mixture was taken (time zero) and stored at 4° C. until required. The Sepabeads 825® broth was then incubated at 35° C. for 44-48 hours and shaken at 180 rpm. At then end of the incubation, an aliquot was taken (time 48) and diluted 1:100 (time 48 d). All aliquots drawn were tested for the presence of the Legionella 16S rRNA gene as described earlier. The detection of a 1-2 log difference between time 48 (or time 48 d) and time zero indicated the presence of viable Legionella in the test sample.

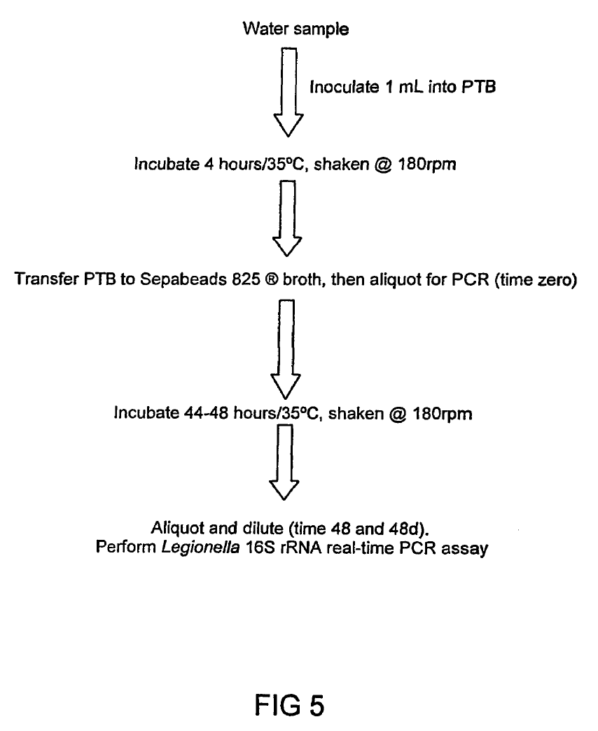

A schematic of the final methodology is shown in FIG. 5.

### Example 9

### Trials with Spiked Environmental Waters

Water from a cooling tower, evaporative tower, and reservoir water were spiked with low level L. pneumophila and L. anisa and tested as described in Example 8, with the inclusion of a heat treatment step as outlined in AS/NZS 3896:1998. Samples were tested in parallel with the current standard method, AS/NZS 3896:1998 and heterotrophic colony counts (HCC) performed according to AS/NZS 4276.3.1. The HCC for the cooling tower and evaporative tower water was >100,000 cfu/mL, and the reservoir water was 500 cfu/mL.

The results are shown in Table 5.

TABLE 5
Heat treated and non-heat treated sample recovery
in Sepabeads ® broth

| | | Inc. | AS/NZS | | |
|---|---|---|---|---|---|
| | | temp | 3896 | | Culture after 48 |
| Sample | Description of water | (° C.) | (cfu/mL) | SP825 | hours |
| 1 | Cooling tower LEPN HEAT TREAT | 35 | 50 | ND | NEG |
| 2 | Cooling tower LEPN NON HEAT TREAT | 35 | 50 | D | POS |
| 3 | Cooling tower LEAN HEAT TREAT | 35 | 70 | ND | NEG |
| 4 | Cooling tower LEAN NON HEAT TREAT | 35 | 70 | D | POS |
| 5 | Evap. tower LEPN HEAT TREAT | 35 | <10 | ND | NEG |
| 6 | Evap. tower LEPN NON HEAT TREAT | 35 | <10 | ND | NEG |
| 7 | Evap. tower LEAN HEAT TREAT | 35 | <10 | ND | NEG |
| 8 | Evap. tower LEAN NON HEAT TREAT | 35 | <10 | ND | NEG |
| 9 | Hope Valley LEPN HEAT TREAT | 35 | 100 | D | POS |
| 10 | Hope Valley LEPN NON HEAT TREAT | 35 | 100 | D | POS |
| 11 | Hope Valley LEAN HEAT TREAT | 35 | 100 | ND | NEG |
| 12 | Hope Valley LEAN NON HEAT TREAT | 35 | 100 | D | OVERGROWN |

D, Detected by real-time PCR
ND, Not Detected by real-time PCR
LEPN, L. pneumophila serogroup 1
LEAN, L. anisa

Samples 1-4 (table 5) represent typical low level Legionella laden water samples. Samples in Sepabeads 825® were only positive for non-heat treated samples. Close scrutiny of parallel culture plates revealed that Legionella was not isolated on the heat treated plates, but only on direct inoculation plates.

A similar trend is observed in samples 9-12. In the case of these samples the AS/NZS 3896 counts were higher than samples 14, and there was 20% recovery of L. pneumophila serogroup 1 on the culture heat treated plates (data not shown), and hence Sepabeads® broth recovery in sample 9. L anisa, however, was negative on culture plates and Sepabeads® broth for heat treated samples, suggesting that L. anisa is more heat sensitive than L. pneumophila and that the effect of heat on Legionella is species and inoculum dependant.

The efficiency of organism removal by PTB, transfer to Sepabeads 825® broth, and detection by real-time PCR is highlighted by the detection of low-level Legionella in relatively contaminated water (HCC >100,000 cfu/mL).

### Example 10

### Compliance with AS/NZS 4659.1:1999 ‘Guide to Determining the Equivalency of Food Microbiology Test Methods-Qualitative Test’

This stage challenged the test method and involved subjecting 5 water matrices (cooling tower water, evaporative water, spa pool water, potable water, and reservoir water) to low level spikes of 5 different strains of Legionella (L. pneumophila sg1 and 6, L. anisa, L. bozemanii, L. micdadei). A non-spiked ‘blank’ sample was run concurrently. Additionally, the samples were tested by the current standard method (AS/NZS 3896:1998).

The final data are presented as suggested in AS/NZS 4569:1999.

| | | INOCULATED | | | UNINOCULATED | |
|---|---|---|---|---|---|---|
| | | PTB/SP825 | | | PTB/SP825 | |
| | | POS | NEG | | POS | NEG |
| AS/NZS | POS | 70 | 0 | POS | 0 | 0 |
| 3896 | NEG | 5 | 0 | NEG | 0 | 75 |

If the inoculated and un-inoculated data set are combined:

PTB/SP825

| | | POS | NEG |
|---|---|---|---|
| AS/NZS | POS | 70 | 0 |
| 3896 | NEG | 5* | 75 |

*AS/NZS 4569: 1998 states that this value must be zero for the test to be accepted. In this case, it proves that the broth is superior to current culture methods as these 5 samples represent low-level Legionella spikes that were detected by the broth method and not the standard culture method (as part of replicate analyses). Additionally, the significance of the disparity was challenged according to the criteria in 4.8 (b) of the standard, and it was found that the difference observed due to this discrepancy was insignificant (McNemar's test).

In summary, these results demonstrate that (i) the broth method detected 5 more low-level Legionella counts than current culture techniques; (ii) the PCR confirmation protocol proved robust for the detection of Legionella as indicated by the absence of true false positives; and (iii) these data confirm the ability of the broth method to reliably detect Legionella in water matrices commonly submitted for analysis.

### Example 11

### Semi-Quantitation of Results

Semi-quantitation can be achieved by performing duplicate analyses on a body of water; specifically, the method be performed as outlined hereinbefore described plus the inclusion of a dilution.

Depending on state, country, and specific legislation, action limits may apply to Legionella counts. As an example, AS/NZS 3666.3 “Performance based maintenance of cooling water systems” has a minimum detection limit of 10 cfu/mL, and an action limit of 1000 cfu/mL for Legionella counts, i.e. there is a 100-fold difference between action and no action limits. The use of this 100-fold difference can be used as a semi-quantitative tool for a body of water should this be required by law or by the clients.

As a specific example, if the true Legionella count in a body of water was 1000 cfu/mL, then by performing the assay an the neat sample and a 100-fold dilution we would expect that both the neat sample and the 100-fold dilution would be positive; thus indicating the original count in the body of water was greater than or equal to 1000 cfu/mL. Similarly, if the true Legionella count in a body of water was 10 cfu/mL, we would expect the neat sample to be positive and the 100-fold dilution to be negative (as the assay cannot detect 0.1 cfu/mL).

Finally, it will be appreciated that various modifications and variations of the methods and compositions of the invention described herein will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are apparent to those skilled in the art are intended to be within the scope of the present invention.

1-67. (canceled)

68. A method of detecting the presence of a viable micro-organism of interest in a sample, the method including the steps of:
(a) providing a sample including a micro-organism of interest and a contaminating micro-organism, wherein the micro-organism of interest has a lag phase greater than that of the contaminating micro-organism;
(b) inoculating the sample into a first liquid medium including an anti-microbial agent that kills actively dividing micro-organisms;
(c) incubating the first liquid medium so inoculated for a period of time sufficient to allow the contaminating micro-organism in the sample to reach log phase but not sufficient for the micro-organism of interest to reach log phase;
(d) inoculating a second liquid medium with an amount of the first liquid medium so incubated, the second medium including an agent that inhibits the activity of the anti-microbial agent;
(e) incubating the second liquid medium so inoculated for a period of time sufficient to allow growth of the micro-organism of interest; and
(f) detecting the presence of viable micro-organisms of interest in the sample by an increase in the value of a first parameter that correlates with the number of micro-organisms of interest detected in the second liquid medium incubated for a period of time to allow growth of the micro-organism of interest, as compared to the value of a second parameter that correlates with the number of micro-organisms of interest detected in any one or more of (i) the sample; (ii) the first liquid medium inoculated with the sample; and (iii) the first liquid medium incubated for a period of time sufficient to allow the contaminating micro-organism in the sample to reach log phase but not sufficient for the micro-organism of interest to reach log phase.

69. A method according to claim 68, wherein the micro-organism of interest is a bacterium.

70. A method according to claim 69, wherein the bacterium is selected from one of the group consisting of a Legionella spp, a bacteria of the genus Mycoplasma, ammonia oxidizing bacteria, Treponema spp., a bacteria of the phylum Spirochaetes, Borrelia spp., Bartonella spp., Afipia spp., Bordatella spp., Brucella spp., Francisella spp., Leptospira spp., Leptonema spp. and Mycobacterium spp.

71. A method according to claim 68, wherein the sample is an environmental sample.

72. A method according to claim 68, wherein the first liquid medium further includes a detoxifying agent.

73. A method according to claim 68, wherein the agent that inhibits the activity of the anti-microbial agent in the second medium is an ion exchange resin.

74. A method of propagating a micro-organism of interest in a sample, the method including the steps of:
(a) providing a sample including a micro-organism of interest and a contaminating micro-organism, wherein the micro-organism of interest has a lag phase greater than that of the contaminating micro-organism;
(b) inoculating the sample into a first liquid medium including an anti-microbial agent that kills actively dividing micro-organisms;
(c) incubating the first liquid medium so inoculated for a period of time of sufficient to allow the contaminating micro-organism in the sample to reach log phase but not sufficient for the micro-organism of interest to reach log phase;
(d) inoculating a second liquid medium with an amount of the first liquid medium so incubated, the second medium including an agent that inhibits the activity of the anti-microbial agent; and
(e) incubating the second liquid medium so inoculated for a period of time sufficient to allow growth of the micro-organism of interest.

75. A method according to claim 74, wherein the micro-organism of interest is a bacterium.

76. A method according to claim 75, wherein the bacterium is selected from one of the group consisting of a Legionella spp, a bacteria of the genus Mycoplasma, ammonia oxidizing bacteria, Treponema spp., a bacteria of the phylum Spirochaetes, Borrelia spp., Bartonella spp., Afipia spp., Bordatella spp., Brucella spp., Francisella spp., Leptospira spp., Leptonema spp. and Mycobacterium spp.

77. A method according to claim 74, wherein the sample is an environmental sample.

78. A method according to claim 74, wherein the first liquid medium further includes a detoxifying agent.

79. A method according to claim 74, wherein the agent that inhibits the activity of the anti-microbial agent in the second medium is an ion exchange resin.

80. A liquid micro-organism growth medium including an ion-exchange resin with either or both of the properties of an average pore radius of less than 200 Angstroms and an average surface area per gram of greater than 600 m²/g.

81. A method of detecting the presence of a viable micro-organism of interest in a sample containing contaminating micro-organisms, the method including the steps of:
(a) using an anti-microbial agent to substantially reduce the number of viable contaminating micro-organisms in the sample without substantially reducing the number of viable micro-organisms of interest in the sample;
(b) inhibiting the activity of the anti-microbial agent after the reduction in the number of viable contaminating bacteria;
(c) propagating the viable micro-organisms of interest after the inhibition of the activity of the anti-microbial agent; and
(d) detecting the presence of viable micro-organisms of interest in the sample by an increase in the value of a first parameter correlated with the number of micro-organisms of interest detected in the sample after propagation, as compared to the value of a second parameter that correlates with the number of viable micro-organisms detected in the sample before propagation of the viable micro-organisms of interest.

82. A method of detecting the presence of a viable micro-organism of interest in a sample containing contaminating micro-organisms, the method including the steps of:
(a) using an anti-microbial agent to substantially reduce the number of viable contaminating micro-organisms in the sample without substantially reducing the number of viable micro-organisms of interest in the sample;
(b) inhibiting the activity of the anti-microbial agent after the reduction in the number of viable contaminating bacteria;
(c) propagating the viable micro-organisms of interest after the inhibition of the activity of the anti-microbial agent; and
(d) detecting the presence of viable micro-organisms of interest in the sample by an increase in the number of micro-organisms of interest detected in the sample after propagation, as compared to the number of viable micro-organisms detected in the sample before propagation of the viable micro-organisms of interest.

83. A method according to claim 82, wherein the viable micro-organisms are detected by culturing.

84. A method according to claim 83, wherein the viable micro-organisms are detected by culturing on a solid medium.

85. A kit for growth and/or detection of a micro-organism, the kit including:
a liquid medium for growth of the micro-organism; and
an ion-exchange resin with either or both of the properties of average pore radius of less than 200 Angstroms and an average surface area per gram of greater than 600 m2/g, the ion exchange resin being separate to the liquid medium or combined with the liquid medium;
the kit optionally further including instructions and/or reagents for the growth and/or detection of the micro-organism.
